(19) United States
(12) Patent Application Publication
(10) Pub. No.: US 2010/0303853 A1
(43) Pub. Date: Dec. 2, 2010

(54) Novel Nacl Salt Substitute Agent, Use Thereof and Products Containing Same

(75) Inventors: Pascal Lejeune, Tourcoing (FR); Alain Simonneau, Paris (FR); Marc Mercier, Vincennes (FR)

Correspondence Address: MCANDREWS HELD & MALLOY, LTD, 500 WEST MADISON STREET, SUITE 3400, CHICAGO, IL, 60661, US

(73) Assignee: LESAFFRE ET COMPAGNIE, Paris (FR)

(21) Appl. No.: 12/739,241

(22) PCT Filed: Oct. 23, 2008
(86) PCT No.: PCT/FR08/51912

§ 371 Date: Apr. 22, 2010

(30) Foreign Application Priority Data
Oct. 26, 2007 (FR) 0758628

(51) Int. Cl. A61K 36/06; A61P 3/00

(52) U.S. Cl. 424/195.16

(57) ABSTRACT

The present invention relates to a novel NaCl salt substitute agent free of aromatic flour and comprising at least one 5′-nucleotide yeast extract, preferably containing at least 1% of 5′-nucleotides relative to the solids content of the yeast extract, and a mixture of low-sodium potassium and ammonium salts, and to the use thereof as a salting agent. It also relates to the use of 5′-nucleotide yeast extract, preferably containing at least 1% of 5′-nucleotides relative to the solids content of the yeast extract, for the off-flavour masking effects thereof and/or for enhancing the salty taste and/or for reducing the amount of low-sodium potassium and/or ammonium salts.

The present invention relates to a novel NaCl salt substitute agent and the use thereof as a salting agent.

For several years there has been a demand for a reduction in the quantity of sodium present in the diet in general, in particular in all the foods identified as important carriers of salts, i.e. the products of bread making and biscuit-making, cooked meats, soups, prepared meals, condiments and sauces. Excessive consumption of sodium could in fact have very harmful consequences for health, and could increase the risk in particular of hypertension. A certain number of public health organizations thus recommend reducing this consumption of sodium in the diet. However, reducing the addition of salt in a food, such as bread doughs and prepared meals and soups, results in tasteless cooked products, generally not well-liked by the consumer.

A certain number of low-sodium salts have thus been identified for their ability to give foods a salty taste. These compounds called low-sodium substitutes are mainly potassium and/or ammonium salts and can be used as a complete or partial substitution for NaCl salt. Nevertheless, with the salty taste being sought, these compounds also provide “off-flavours”, i.e. undesirable aftertastes or spurious flavours. These “off-flavours” have largely been described in the literature as metallic, bitter and soapy aftertastes. These low-sodium substitutes are thus often perceived as unpleasant and nasty by the consumer and can even greatly impair the taste and flavour of the foods that contain them. This disadvantage therefore limits their uses as NaCl salt substitutes, even though public health organizations strongly recommend their use. Such low-sodium substitutes as they are thus do not offer a fully satisfactory solution for reducing sodium in the diet.

Several publications have already described this problem and have attempted to solve it. Thus, it has been proposed to combine low-sodium salts, in particular potassium chloride, with lactose or dextrose (U.S. Pat. No. 3,860,732), fumaric acid (U.S. Pat. No. 3,505,082) or also a mixture of magnesium chloride and magnesium sulphate (WO98/53708).

It has also been proposed to combine low-sodium salts with autolyzed yeasts. The publications U.S. Pat. No. 4,297,375 and EP0103994A1 (Standard Oil Company) have thus proposed the use of autolyzed yeast to reduce the bitterness of the ammonium or potassium chlorides. Nevertheless, such a solution has disadvantages. Firstly, it requires large quantities of autolyzed yeast to be implemented. In addition, it is known that autolyzed yeast provides aromatic notes typical in particular of chicken, meat and cheese. If such aromatic notes are actually capable of masking the perception of the bitterness of low-sodium salts, they nevertheless limit the use of the autolysates to certain particular applications, such as chicken broths, or sausages, as is the case in these publications. Moreover, with the exception of the flavour-enhancing effect of the autolysate, no salty effect peculiar to autolyzed yeast is mentioned. A certain number of these disadvantages are also mentioned and accepted in these publications U.S. Pat. No. 4,297,375 and EP0103994A1.

In a previous patent application WO2005/087013, the Applicant had described a salting and taste-enhancing agent comprising a combination of a yeast extract with acid fermented flour. Nevertheless, if this agent can easily be used in products already requiring a certain quantity of flour, in particular bread making products, it is often desirable to have available an agent that can be more easily formulated and is therefore versatile. The presence of flour in fact sometimes complicates the formulation of the salt substitute agent and can limit the use thereof to only certain applications.

There was therefore a need to have available a salt substitute agent which can be easily formulated in order to be able to be used in broad applications and which may be capable at the same time of providing an intense salty taste without bringing the disadvantages mentioned previously.

The Applicant has just discovered, in a particularly unexpected and surprising manner, that it was possible to produce a salt substitute agent which is free of flour by combining a yeast extract and 5′nucleotides with a mixture of low-sodium ammonium and potassium salts.

The present invention therefore relates to a novel NaCl salt substitute agent comprising a mixture of potassium salts and ammonium salts, and at least one yeast extract and 5′nucleotides, said 5′nucleotides being able to be provided entirely or partially by the yeast extract and being present in a quantity of at least 1% by weight relative to the total weight of the yeast extract, and free of aromatic flour and the use thereof as a salting agent. It also relates to the use of yeast extract and 5′nucleotides to enhance the salty taste of mixtures of potassium and ammonium salts and/or to mask the off-flavours thereof, without adding aromatic flour.

The present invention thus provides a novel salt substitute agent free of aromatic flour comprising at least one yeast extract, 5′nucleotides, preferably in a content of at least 1% of 5′nucleotides relative to the solids content of the yeast extract, and a mixture of low-sodium ammonium and potassium salts. Such an agent is remarkable in that it provides an effect of masking the off-flavours of low-sodium salts while providing a salty taste with an enhanced intensity, without the disadvantages mentioned previously, in particular the aromatic notes typical of chicken, meat and cheese, while being able to be easily formulated.

Such an effect is particularly surprising in the light of current available knowledge on these different components. In fact, this salting agent free of the taste and perceptible flavour of yeast extracts and 5′nucleotides nevertheless allows the off-flavours of the low-sodium substitutes to be masked. Without being bound by an explanatory hypothesis of this effect, it would appear that ammonium salt, in particular NH₄Cl, masks the well-known “umami” effect of yeast extracts and 5′nucleotides in a very unexpected manner. A mixture containing yeast extract, 5′nucleotides had thus been described in the literature, in particular in the patent application FR2264493 and, added to potassium chloride, is described as a useful mixture for seasoning due to its meat flavour in particular “a flavour similar to the flavour of beef or hock extract” that it provides to foods. This is the result of an “umami” effect described in the literature as a taste-enhancing effect due to the presence of glutamic acid. Thus and in a particularly surprising manner, the product according to the invention has an “umami” effect with a weaker intensity than that of a mixture of yeast extract and 5′nucleotides, which makes it very easy to use. Thus, the present invention allows an agent to be produced which gives an intense salty taste provided at the same time by the low-sodium salt and enhanced by the yeast extract and the 5′nucleotides. Moreover, the yeast extract and the 5′nucleotides mask the off-flavours of the low-sodium substitutes, which allows a salt substitute agent having a pleasant flavour to be obtained without impairing either the perception of the taste or the flavour of the foods that contain them. Moreover, the agent according to the invention has an “umami” effect which is lessened by the yeast extracts and 5′nucleotides.

The yeast extract, the 5′nucleotides and the mixture of low-sodium ammonium and potassium salts are compounds that are soluble in water, which allows the agent to be used and formulated very easily and makes it possible to use it as a salt substitute agent in a very large number of applications.

Moreover, the present invention offers the undeniable advantage of combining with the low-sodium substitutes a yeast extract and 5′nucleotides, in particular when the latter derive from yeast, which are natural products and benefit from a better image among consumers.

Moreover, thanks to the present invention, the low-sodium salts content of foods can also be reduced.

The present invention is particularly advantageous in that it offers, moreover, an agent for substituting table, common or cooking salt, which can be used in domestic or industrial applications, as it is or incorporated into food compositions. It therefore allows the quantity of sodium in the diet to be greatly reduced or even completely eliminated, and this without reducing the consumer's organoleptic perception. The present invention therefore provides a response that perfectly corresponds to the recommendations prescribed by public health organizations and to the choice criteria of consumers.

Moreover, the present invention offers a response that is particularly suitable for enabling the administration of potassium salts orally in the treatment of hypokalemia. Through the different combinations of salts that it proposes, the invention moreover makes it possible to control and adapt the intake of potassium, magnesium and ammonium salts depending on the needs of the consumer.

An object of the present invention is therefore a salt substitute agent free of aromatic flour and comprising a mixture of potassium salts and ammonium salts, and at least one yeast extract and 5′nucleotides, said 5′nucleotides being able to be provided entirely or partially by the yeast extract and the 5′nucleotides being present in a quantity of at least 1% by weight relative to the total weight of the yeast extract. Preferably, the mixture of low-sodium salts is a mixture of ammonium chloride and potassium chloride.

By “salt substitute agent” is meant, according to the present invention, an agent capable of being used for its salting effect by entirely or partially replacing NaCl salt and thus reducing the intake of sodium.

The yeast extracts are known products. As previously indicated, they are commonly used as a taste enhancer. By yeast extract is meant, according to the invention, the soluble fraction obtained after enzyme hydrolysis of yeast cells preferably belonging to the Saccharomyces genus. Also according to the invention, the yeast extract is preferably the soluble fraction obtained after autolysis of said yeast cells, i.e. after enzyme hydrolysis performed solely by the endogenous enzymes of yeast. The hydrolysis of the yeast cells can also be carried out by using exogenous enzymes, i.e. by adding additional enzymes, such as in particular proteases.

Preferably, the yeast extract is separated from the insoluble part of the yeast cells. The yeast extract thus separated from the insoluble part offers the advantage of a better preservation without the occurrence of aromatic notes due to oxidation of the membranous lipids of the insoluble part.

According to the invention, the yeast extract preferably belongs to the Saccharomyces genus and also preferably belongs to the Saccharomyces cerevisiae species, including the one called Saccharomyces carlsbergensis. Said Saccharomyces cerevisiae yeast cells are also often called Saccharomyces carlsbergensis when these are brewer's yeast, the precise taxonomic name being Saccharomyces cerevisiae according to “THE YEASTS, a taxonomic study”, 3″ edition, edited by N. J. W. Kreger van Rij-1984 (by contrast, according to the 4^{th} edition of this work from 1998, Saccharomyces carlsbergensis has two synonyms Saccharomyces cerevisiae and Saccharomyces pastorianus, it is the 3^{th} edition of this work dated 1984 which is taken as reference in the present document). According to the invention, the yeast extract is chosen from brewer's or baker's yeast extracts.

Brewer's yeast extracts are generally characterized by the presence of a detectable quantity of humulones, a quantity which, preferably, it is desirable to have as small as possible. Said yeast extract of the agent according to the invention can in particular contain and/or be a brewer's yeast extract, this brewer's yeast extract preferably being debittered, this debittering being able to be carried out in advance by usual and well-known techniques.

The yeast extract contained in the agent according to the invention is preferably a yeast extract obtained without adding salt. Preferably, when the yeast extract comprises or is a brewer's yeast extract, the brewer's yeast extract will be assessed by a tasting panel as very slightly bitter. Preferably, the yeast extract contained in the agent according to the invention will be assessed by such a tasting panel as not having or having little notes of the type “milky”, “buttery”, “cheese” and generally as having a neutral taste profile without a marked note.

Preferably, the yeast extract is present in the form of a dry extract.

The agent according to the invention contains 5′nucleotides, preferably in a content of at least 1% of 5′nucleotides relative to the solids content of the yeast extract. The nucleotides are phosphoric esters of nucleoside, necessarily comprising:

a purine or pyrimidine nitrogenous base,

a pentose: ribose or deoxyribose, and

one or more phosphoric acid molecules.

In the context of the present invention, the term “5′-nucleotides” is meant to include 5′-nucleotides as they are, as well as their salts, hydrates, hydrated salts, and other physiologically acceptable forms. In particular, the 5′nucleotides comprise at least one 5′phosphate group. According to the invention, the 5′-nucleotides are or advantageously comprise ribonucleotides. In a useful manner, the 5′-nucleotides, and in particular the 5′-ribonucleotides, are or comprise 5′-nucleotide monophosphates, preferably 5′-IMP (inosine 5′-monophosphate), 5′-GMP (guanosine 5′-monophosphate), 5′-AMP (adenosine 5′-monophosphate), 5′-UMP (uridine 5′-monophosphate) or 5′-CMP (cytidine 5′-monophosphate).

According to a preferred embodiment of the invention, the agent contains at least 1% of 5′nucleotides relative to the solids content of the yeast extract. Also preferably, the agent contains 5 to 40%, and advantageously 10 to 20%, of 5′nucleotides defined relative to the solids content of the yeast extract.

The 5′nucleotides can be provided as purified components or be provided by the yeast extract. For the present invention, the 5′-nucleotides can be obtained by any one of the known production and synthesis methods described in the literature.

Very advantageously, the yeast extract according to the invention is naturally rich in 5′nucleotides and/or can be enriched by adding a quantity of 5′nucleotides, preferably obtained from yeast.

It should be noted that in the conventional methods for producing yeast extracts which comprise an autolysis or an enzyme hydrolysis, essentially by the endogenous enzymes of yeast, the ribonucleic acid (RNA) of yeast is mainly decomposed into tasteless nucleotides such as adenosine 3′-monophosphate (3′-AMP) and guanosine 3′-monophosphate (3′-GMP), into bases, riboses and phosphates. These endogenous enzymes of yeast are essentially ribonucleases, specific 3′-nucleotidases, acid phosphatases. These conventional methods do not therefore allow yeast extracts rich in 5′-nucleotides to be obtained. Methods that are suitable for obtaining yeast derivatives rich in 5′-nucleotides are known and described for example in the reference work “Yeast Technology” by G. Reed and T. W. Nagodawithana, 2nd edition (Van Nostrand Reinhold, ISBN 0-442-31892-8) pages 382 to 385.

It is in particular known to prepare yeast extracts rich in 5′-nucleotides by enzyme hydrolysis of yeasts in the presence of 5′-phosphodiesterase with deactivation of the phosphatases and endogenous nucleases of the yeast. Yeast extracts containing the following 5′-nucleotides are thus obtained: 5′-GMP, 5′-UMP, 5′-CMP and 5′-AMP (corresponding to the four RNA bases). 5′-AMP can also be converted to a 5′-IMP compound that has a stronger smell and enhances the taste more by adding AMP deaminase. A yeast extract containing 5′-GMP, 5′-UMP, 5′-CMP, 5′-IMP, and optionally also a residual quantity of 5′-AMP, is thus obtained. This conversion allows the smell and/or taste-enhancing effect to be strengthened if desired.

From the known methods suitable for producing yeast derivatives rich in 5′-nucleotides, the following methods can also be cited:

U.S. Pat. No. 4,810,509 describes a method for producing yeast extract rich in 5′-nucleotides comprising (1) a stage of heating a yeast suspension to between 55° C. and 70° C., (2) a stage of autolyzing the yeast cells at pH 8 to 10, (3) adjusting the pH of the suspension of autolyzed yeasts to between 5 and 7, (4) a stage of heating this suspension to 90° C. or more, (5) eliminating the insoluble material of this heated suspension and (6) recovering the yeast extract containing 5′-nucleotides. A treatment carried out after the autolysis with the 5′-adenylate deaminase enzyme (=AMP deaminase) allows the 5′-AMP to be converted to 5′-IMP for a stronger smell effect.

The method according to EP-A-0299078 consists of heating a yeast suspension containing a large quantity of RNA to between 80° C. and 120° C. (destroying the ribonucleases), then extracting the RNAs with an alkaline treatment and decomposing them into 5′-nucleotides by the action of a 5′-phosphodiesterase, and optionally a deaminase in order to obtain an extract rich in 5′-GMP and 5′-IMP.

The method according to WO02/067959 consists in preparing a yeast derivative by an autolysis carried out at a temperature greater than 35° C., for example comprised between 35-70° C., preferably between 50-60° C. The yeasts are preferably hydrolyzed during or after the autolysis with one or more proteases. Optionally, the product can be centrifuged and an additional ultrafiltration stage for the supernatant can be carried out.

According to the invention, by “yeast extract rich in 5′nucleotides” is meant a yeast extract naturally rich in or enriched by 5′nucleotides. Preferably, such an extract contains at least 1% of 5′nucleotides relative to the solids content of the yeast extract and preferably 5 to 40% of 5′nucleotides, also advantageously 10 to 25%. A significant salting effect is obtained in a very advantageous manner with baker's yeast extracts containing at least 10% of 5′nucleotides in solids relative to the yeast extract.

According to the invention, the NaCl salt substitute agent and/or the compositions do not contain “aromatic flour”, i.e. a flour made aromatic by heating or an intrinsically aromatic flour. According to the invention, by “intrinsically aromatic flour” is meant an aromatic flour naturally containing aromatic notes, without a heating stage being necessary. Such flours are in particular germ flours, in particular wheat, bran, chestnut, buckwheat, quinoa and teff flours. According to the invention, by “flour made aromatic by heating” is meant a flour obtained by a method comprising a heating stage. This heating stage promotes Maillard reactions and allows the aromatic notes that are sought to be obtained. The overdried flours obtained with a heating stage with a duration and/or intensity that is insufficient to develop aromatic notes do not constitute an aromatic flour. The flours made aromatic by heating are characterized by an EBC value greater than or equal to 8 when this derives from a flour with an initial EBC value strictly lower than 3. When the flour made aromatic by heating derives from a flour with an initial EBC value equal to or greater than 3, it is characterized by an increase in this EBC value of 10 points or more. The EBC (European brewery convention) value is determined by a known and standardized method called Analytica EBC No. 4.7.2, applied in a standard fashion in brewery for measuring the colour of malts. The flour made aromatic by heating is often described in the literature as “grilled”, “toasted” or “roasted”. It is in particular roasted malted or acid fermented flours.

According to the invention, the NaCl salt substitute agent and/or the compositions in which it is used do not contain roasted malted flours, nor acid fermented flours, nor wheat-germ flours, and/or bran flours. Preferably, the NaCl salt substitute agent and/or the compositions in which it is used do not contain flour.

The agent according to the invention contains at least one mixture of low-sodium potassium and ammonium salts. Potassium chloride (KCl) and/or ammonium chloride (NH₄Cl) constitute the preferred salts according to the present invention.

Ammonium chloride is particularly advantageous because of the strong salting power that it provides Ammonium salts, and ammonium chloride in particular, surprisingly allow the taste-enhancing effect called the “umami” effect of the yeast extracts and 5′nucleotides to be reduced. A particularly advantageous mixture according to the invention is a mixture in which the ratio of ammonium salts/potassium salts, and preferably NH₄Cl/KCl, by weight is comprised between 0.05 and 1.5. It has been noted, according to an advantageous embodiment, that the salty intensity is optimum and the perception of off-flavours, in particular the perception of bitter notes, is minimal for NH₄Cl/KCl ratios by weight comprised between 0.05 and 0.66, preferentially between 0.07 and 0.25, and preferably approximately 0.1. Such a mixture is particularly appropriate for bread making applications.

Larger doses of ammonium salts, in particular of NH₄Cl, allow the salty intensity to be increased for certain food preparations where a very intense salting effect is desired, in particular in seasonings, prepared meals, soups, processed industrial products, typically snacks, crackers, crisps, cocktail biscuits. Another particularly advantageous mixture according to the invention is thus a mixture with an NH₄Cl/KCl ratio by weight which is comprised between 0.4 and 1.5, preferentially between 0.7 and 1.4 and preferably approximately 1.3.

Particularly advantageously, the salt substitute agent according to the invention will be free of aromatic flour and will be constituted exclusively by yeast extract rich in 5′nucleotides and a mixture of potassium and ammonium salts, also preferably ammonium chloride (NH₄Cl), potassium chloride (KCl), and also preferably in an NH₄Cl/KCl ratio comprised between 0.5 and 1.5.

The salt substitute agent and the compositions according to the invention can moreover contain a certain quantity of sodium depending on requirements. According to a particular embodiment, the salt substitute agent and the compositions according to the invention will be completely free of added sodium. Such an agent is particularly useful for being added to a food composition already containing sodium and/or for reducing the sodium content of said composition.

According to another embodiment of the invention, the salt substitute agent contains at least 20% of sodium, in particular in NaCl form, by weight of the agent, preferably between 30% and 60% by weight of the agent. An intense salty taste was obtained with a 50% sodium content by weight of the agent and constitutes a preferred embodiment of the invention, in particular for the use thereof as a substitute for common salt NaCl. The sodium can be provided, in part or entirely, by the yeast extract rich in 5′nucleotides. Such an agent is particularly appropriate for being used as the only source of sodium in a food composition.

The agent according to the invention advantageously has a ratio by weight between the solids of yeast extract rich in 5′nucleotides and the solids of the low-sodium salts comprised between 10 and 50% and preferably from 15% to 30%.

The salt substitute agent according to the invention can be present in any form suitable for the use thereof in human and/or animal diet. It is preferably present in dry form. According to a first embodiment, it is present in the form of a simple mixture of the components in dry form. Advantageously, the salt substitute agent is obtained by preparing a yeast extract rich in 5′nucleotides in liquid form, then adding the mixture of low-sodium potassium and ammonium salts, preferably potassium chloride and ammonium chloride. The agent in liquid form obtained in this way can be used as it is or dried, preferably by atomization. Advantageously, the salt substitute agent is therefore in a dry powder form comprising the different components. The other components of interest, in particular succinic acid, can be added between these different stages of preparing the agent, and in particular by incorporation in the salt substitute agent in liquid form before drying or by incorporation in the salt substitute agent after drying. The choice will be made depending on the liquid or dry presentation of these other components and their resistance to drying.

According to a second embodiment, the salt substitute agent according to the invention is present in the form of a product of identical or similar presentation to common salt NaCl, i.e. homogeneous and of the same granulometry as NaCl salt in the form of fine salt, coarse salt or fleur de sel. The size of the granules will thus be advantageously comprised between 100 and 3000 microns in diameter, preferably between 200 and 2000 microns in diameter. These characteristics can be obtained by implementing standard techniques and methods known in the field of formulation, in particular coating, crystallization or agglomeration techniques. The crystal of the mixture of low-sodium potassium and ammonium salts can thus be coated by the yeast extract rich in 5′nucleotides. It is also possible to use a carrier, for example NaCl, and to coat it with a yeast extract mixture rich in 5′nucleotides and low-sodium potassium and ammonium salts. According to an alternative, the components of the agent are solubilized then simultaneously crystallized in such a way that the yeast extract rich in 5′nucleotides may be trapped in the mixture of low-sodium salts. The dry products constituting the agent can also be aggregated together.

The salt substitute agent can also contain other compounds of interest. These can in particular be compounds necessary for formulating the agent in the desired form. Such compounds will preferably be free of taste or perceptible flavour capable of impairing the organoleptic qualities of the salt substitute agent and/or the compositions in which said agent is capable of being used. By way of example, these can be additives such as texturizing agents such as microcrystalline cellulose, dextrose or magnesium stearate, adjuvants, anti-agglomerating agents such as calcium silicate (E552), anticaking agents such as wheat fibres, or technological additives, bulking agents such as maltodextrin. The maltodextrin can be present in a quantity of at least 50% by weight relative to the weight of the composition. These can also be compounds which have a use in terms of public health, such as fluorine and iodine or other salts such as magnesium salts in particular magnesium chloride (MgCl₂) and magnesium sulphate. Such a mixture offers the advantage of compensating for dietary deficiencies of magnesium and potassium, in compliance with the recommendations of public health organizations.

According to a particularly advantageous embodiment, the salt substitute agent contains succinic acid, preferably at least 0.2% by weight of the agent, also preferably in a content comprised between 0.5 and 5% by weight, and advantageously 1%. It was observed that the succinic acid very advantageously allows the perception of the salty taste of the salt substitute agent according to the invention to be accelerated. The salty taste is thus perceived more rapidly by the consumer.

An object of the invention is the use of the agent according to the invention to entirely or partially substitute NaCl salt. The salt substitute agent can be used as a product as it is for its salting effect, i.e. to entirely or partially replace household salt NaCl. In this last case, it will preferentially contain between 20 and 60% of sodium, preferably approximately 50% by weight of the agent. It can also be added for its salting effect in a composition according to the invention intended for human and/or animal diet already containing sodium or not. According to the invention, the agent can be used to reduce the quantity of sodium, or even to eliminate it, in human and/or animal diet.

An object of the present invention is therefore a method for producing a composition intended for human and/or animal diet consisting of replacing some or all of the quantity of sodium chloride with the salt substitute agent according to the invention. Advantageously, the relative sodium content will be reduced by at least 25%, and/or the absolute sodium content is preferably approximately 50%. Advantageously, the relative sodium content will be reduced by at least 0.12 g per 100 g of product. Preferably, the composition will be prepared without adding any sodium salts and will have a sodium content reduced by half relative to the common compositions of the same nature and less than 0.2 g, preferably less than 0.02 g, and also preferably less than or equal to 0.01 g, per 100 g of product.

The present invention is therefore also relates to any composition intended for human and/or animal diet containing said salt substitute agent. This can be in particular bread and/or bakery product, cooked meat, seasoning products, prepared meals, soups, processed industrial products, typically snacks, crackers, crisps, cocktail biscuits, etc. and any other cooked or uncooked complex food preparation. Advantageously, these ready-to-eat compositions will contain less than 0.12 g of sodium per 100 g or 100 ml of product, preferentially less than 0.04 g, and preferably less than 0.02 g, still more preferably less than 0.005 g of sodium, per 100 g or 100 ml of product.

According to another advantageous embodiment, the compositions intended for human and/or animal diet containing said salt substitute agent contain 0.14 g or less than 0.14 g of sodium per consumed portion (“per labelled serving” “per referenced amount”) and preferably 0.005 g or less than 0.005 g of sodium per consumed portion.

Bread improvers and bread making products such as bread doughs, breads and other cooked bread making products, free of aromatic flour can be cited as preferred compositions according to the invention. Such products are particularly suitable within the framework of a low-sodium diet.

Among the bakery products free of aromatic flour, bread doughs, deferred bread making products, in particular deep-frozen raw dough rolls, raw products called “pre-proofed”, raw products called “proof-blocked”, and also pre-cooked, deep-frozen dough rolls which can be obtained by fermentation, pre-cooking and deep-freezing a dough free of aromatic flour according to the invention can be cited.

They can also be cooked bakery products free of aromatic flour and capable of being obtained by the fermentation and cooking of an above-mentioned dough, such as breads, preferably baguettes, viennoiseries and/or brioches. Preferably, the cooked bread making products according to the invention are common French breads or at least French type breads such as baguettes.

The salt substitute agent according to the invention can be present in the form of a bread improver. A bread improver according to the invention can be dry or liquid and can also comprise one or more ingredients having an improving effect, and in particular one or more ingredients chosen from the group consisting of ascorbic acid, emulsifiers, stabilizer-thickeners and enzymes. The improver according to the invention can thus comprise one or more ingredients having an improving effect, such as ascorbic acid, L-cysteine, or deactivated yeast, stabilizer-thickeners such as pre-gelatinized flour, modified starches, CMC (carboxymethyl cellulose), gums such as for example xanthan gum, algae extracts such as alginates or carrageenans, or a combination of these different stabilizer-thickeners, emulsifiers, such as for example lecithin or mono- and diglycerides of fatty acids or diacetyl tartaric esters of mono- and diglycerides of fatty acids, etc., or also a combination of one or more emulsifiers such as those mentioned previously, enzymes, such as for example amylases, and in particular alpha-amylases, including for example maltogenic alpha-amylases or other anti-staling alpha-amylases, hemicellulases, and in particular xylanases, glucose oxidases, amyloglucosidases, lipases, phospholipases, etc., non-aromatic cereal flours or other ingredients typical of the composition of special breads.

When the dry improver is presented in the form of powder, this support can be, for example, dried flour.

In bread making, the invention is not limited to these applications of the salt substitute agent according to the invention and includes all of the methods, all of the doughs and all of the products of bread making and uses comprising the novel salt substitute agent for bread making according to the invention and free of aromatic flour.

Compositions according to the invention can also be other foods often considered as carrying the most sodium, namely cooked meats, soups, cheeses, condiments and sauces, seasoning products in general, prepared meals, processed industrial products, typically snacks, crackers, crisps, cocktail biscuits, etc.

More generally, another object of the present invention is therefore the use of yeast extract and 5′nucleotides, and preferably at least 1% relative to the solids content of the yeast extract, in combination with a mixture of low-sodium potassium and ammonium salts, without adding aromatic flour. According to a preferred embodiment, the nucleotides are preferably provided by the yeast extract and the invention then relates to the use of yeast extract rich in 5′nucleotides in combination with a mixture of low-sodium potassium and ammonium salts. This use allows in particular the salty intensity to be increased and/or the off-flavours of this mixture of low-sodium salts, preferably ammonium and potassium chlorides, to be masked. The yeast extract and 5′nucleotides, preferably in the form of yeast extract rich in 5′nucleotides, can thus be added into products already containing this mixture of low-sodium potassium and ammonium salts. According to the invention, the yeast extract and 5′nucleotides, preferably in the form of a yeast extract rich in 5′nucleotides, can also be used to reduce the quantity of low-sodium potassium and ammonium salts. Thus, the invention relates to a method for masking the off-flavours of a mixture of low-sodium potassium and ammonium salts in a food composition and/or enhancing the intensity of the salty taste of a mixture of low-sodium potassium and ammonium salts, preferably of a mixture of potassium and ammonium chlorides, comprising the addition of yeast extract and 5′nucleotides, preferably in the form of yeast extract rich in 5′nucleotides, without adding aromatic flour. It also relates to a method for reducing the quantity of low-sodium potassium and ammonium salts, preferably of a mixture of potassium and ammonium chlorides in a food composition, comprising the preparation of a food composition containing a reduced quantity of said low-sodium salts and the addition of a yeast extract and 5′nucleotides, preferably in the form of yeast extract rich in 5′nucleotides, without adding aromatic flour, this addition allowing the reduction in the quantity of low-sodium salts to be compensated for while preserving the same salty taste. Moreover, the present invention relates to a product or kit comprising a yeast extract and 5′nucleotides, preferably in the form of yeast extract rich in 5′nucleotides, and a composition comprising a mixture of low-sodium potassium and ammonium salts, preferably a mixture of potassium and ammonium chlorides, the yeast extract and the 5′nucleotides being in a quantity large enough to produce the appropriate salty taste and/or to mask the off-flavours of the mixture of low-sodium salts, without adding aromatic flour. It also relates to a product or kit comprising yeast extract and 5′nucleotides, preferably in the form of a yeast extract rich in 5′nucleotides, and a composition comprising a mixture of low-sodium potassium and ammonium salts, preferably a mixture of potassium and ammonium chlorides, for a simultaneous use allowing the off-flavours of the mixture of low-sodium salts to be masked and/or the quantity of the mixture of low-sodium salts to be reduced, while preserving the same salty taste and/or intensifying the salty taste, without adding aromatic flour.

Another object of the present invention is therefore a method for producing a composition intended for human and/or animal diet, containing a mixture of low-sodium potassium and ammonium salts, preferably a mixture of potassium and ammonium chlorides, consisting of adding a yeast extract and 5′nucleotides, preferably in the form of yeast extract rich in 5′nucleotides, and/or replacing part of said mixture with a yeast extract and 5′nucleotides, preferably in the form of a yeast extract rich in 5′nucleotides, without adding aromatic flour.

According to the invention and unless otherwise indicated, the term “composition” designates a composition containing at least the yeast extract and 5′nucleotides, preferably in the form of yeast extract rich in 5′nucleotides, and at least one mixture of low-sodium potassium and ammonium salts, preferably a mixture of potassium and ammonium chlorides but not containing aromatic flour, and preferably completely free of flour.

Another object of the present invention is the salt substitute agent containing at least the yeast extract and 5′nucleotides, preferably in the form of yeast extract rich in 5′nucleotides, and a mixture of low-sodium potassium and ammonium salts, preferably a mixture of potassium and ammonium chlorides, as a medicament, in particular for orally treating hypokalemia, without adding aromatic flour.

The following examples are provided to illustrate the invention and should in no way be considered as limiting the scope of the invention.

### EXAMPLES OF SALT SUBSTITUTE AGENTS ACCORDING TO THE INVENTION

The salt substitute agents according to the invention detailed below were produced by simply mixing the following components:

The yeast extract present in the dry agent according to the invention is an autolysate of a baker's yeast belonging to the Saccharomyces cerevisiae species containing at least 10% of 5′nucleotides in solids relative to the yeast extract, manufactured with or without the addition of NaCl salt. A yeast extract of the Springer 2000 range sold by BIO SPRINGER, from Maisons-Alfort, France was more specifically used in these examples.

Substitute Agent 1:

| | in % by weight of |
|---|---|
| | the composition |
| yeast extract | 26 of which NaCl 6 |
| 5′nucleotides | 3 |
| KCl | 65 |
| NH₄Cl | 6 |

This agent is particularly suitable for producing bread with a reduced salt content and can be incorporated at 2.53% (baker percentages) in the flour according to the following recipe:

| | |
|---|---|
| flour | 100 |
| KCl | 1.12 |
| NH₄Cl | 0.11 |
| yeast extract | 0.34 |
| 5′nucleotides | 0.06 |
| NaCl | 0.9 |

to produce a bread dough, without adding another source of sodium.

Substitute Agent 2:

| | in % by weight of |
|---|---|
| | the composition |
| yeast extract | 16 |
| 5′nucleotides | 4 |
| KCl | 73 |
| NH₄Cl | 7 |

This agent is particularly suitable for producing bread without sodium and can be incorporated at 1.53% (baker percentages) in the flour according to the following recipe:

| | |
|---|---|
| flour | 100 |
| KCl | 1.12 |
| NH₄Cl | 0.11 |
| yeast extract | 0.24 |
| 5′nucleotides | 0.06 |

to produce a bread dough, without adding sodium.

A panel of experts tested these 2 agents on their own or included in bread doughs and noted an intense salting effect, a pleasant flavour without impairing the taste of the breads that contain them.

Moreover, a decrease in the off-flavours of the low-sodium salts and the umami effect of the yeast extract and 5′nucleotides was observed.

Substitute Agent 3:

| | in % by weight of |
|---|---|
| | the composition |
| yeast extract | 24 |
| 5′nucleotides | 6 |
| KCl | 30 |
| NH₄Cl | 39 |
| Succinic acid | 1 |

This agent is particularly suitable for providing an intense salting effect, in particular as a substitute for part of the sodium in preparations where a strong salty intensity is desired.

Test 1:

A panel of experts tested this agent in water and in the contents indicated in the following recipes:

| | Recipe 1 | Recipe 2 | Recipe 3 |
|---|---|---|---|
| | Control | dosage 1/1 | dosage 0.6/1 |
| Ingredients | in % by weight | in % by weight | in % by weight |
| NaCl salt | 0.5 | 0.25 | 0.25 |
| SubstituteAgent 3 | / | 0.25 | 0.15 |

An intense salting effect, without off-flavours or exaggerated umami effect, was observed in these 3 recipes. In particular, recipes 1 and 2 were considered as having a very similar salty intensity. The intensity of recipe 3 was identified as less salty than that of recipes 1 and 2 but always devoid of off-flavours.

Test 2:

| The following mixture | in % by weight of |
|---|---|
| was produced: | the composition |
| sugar | 14 |
| monosodium glutamate | 17 |
| chicken flavours | 8 |
| garlic powder | 3 |
| chicken fat | 5 |
| turmeric | 0.80 |
| white pepper | 0.80 |
| maltodextrin | 51.4 |
| NaCl salt | 0 |

This mixture was incorporated in water at 1.5% by weight to constitute a stock base.

The salting agents were then added in the contents indicated in the following recipes (dosage in % by weight):

| | Recipe 1 | Recipe 2 | Recipe 3 |
|---|---|---|---|
| | Control | dosage 1/1 | dosage 0.6/1 |
| Ingredients | in % by weight | in % by weight | in % by weight |
| Broth base | 1.5 | 1.5 | 1.5 |
| NaCl salt | 0.5 | 0.25 | 0.25 |
| Substitute Agent 3 | / | 0.25 | 0.15 |

For these 3 recipes, a panel of experts noted a very intense salting effect and a pleasant flavour without impairing the taste of the stocks that contain them.

1. A salt substitute agent free of aromatic flour, wherein the agent comprises:
a mixture of potassium salts and ammonium salts, and
at least one yeast extract and 5′nucleotides, said 5′nucleotides being able to be provided entirely or partially by the yeast extract, and in that the 5′nucleotides are present in a quantity of at least 1% by weight relative to the total weight of the yeast extract.

2. An agent according to claim 1, wherein the potassium salt is potassium chloride and/or the ammonium salt is ammonium chloride.

3. An agent according to claim 1, wherein the mixture of low-sodium salts has a ratio of ammonium salts/potassium salts, and preferably an NH₄Cl/KCl ratio, comprised between 0.05 and 1.5.

4. An agent according to claim 1, wherein the 5′nucleotides are present at between 5 and 40% relative to the dry solids content of the yeast extract.

5. An agent according to claim 1, wherein the ratio by weight between the dry solids of the yeast extract and the 5′nucleotides on the one hand and the dry solids of the mixture of low-sodium salts on the other hand is comprised between 10% and 50%.

6. An agent according to claim 1, in which the yeast extract is chosen from baker's or brewer's yeast extracts.

7. An agent according to claim 1, wherein the agent is present in dry form.

8. An agent according to claim 1, wherein the agent further comprises sodium, preferably in NaCl form, in a quantity equal to zero or equal to at least 20% by weight of sodium, and preferably 30 to 60% by weight of sodium.

9. An agent according to claim 1, containing at least 0.5% by weight, preferably 0.5% to 5% by weight, of succinic acid.

10. (canceled)

11. A method for masking the off-flavours of a mixture of low-sodium potassium and ammonium salts, preferably potassium and/or ammonium chlorides, and/or for increasing the salty intensity, said method comprising the step of adding a yeast extract and 5′nucleotides, and preferably at least 1% of 5′nucleotides relative to the dry solids content of the yeast extract, without adding aromatic flour.

12. A method for reducing the quantity of a mixture of low-sodium potassium and ammonium salts, preferably potassium and/or ammonium chlorides, said method comprising the step of adding a yeast extract and 5′nucleotides, and preferably at least 1% of 5′nucleotides relative to the dry solids content of the yeast extract, without adding aromatic flour.

13. A composition intended for animal and/or human diet containing the salt substitute agent according to claim 1 free of aromatic flour.

14. A bread improver or bread making product free of aromatic flour containing the agent according to claim 1.

15. A salt substitute agent according to claim 1 as a medicament, the potassium salt being potassium chloride.

16. A method of treating hypokalemia orally by administering an agent according to claim 1, the salt being a potassium chloride.

17. An agent according to claim 2, in which the yeast extract is chosen from baker's or brewer's yeast extracts.

18. An agent according to claim 3, in which the yeast extract is chosen from baker's or brewer's yeast extracts.

19. An agent according to claim 4, in which the yeast extract is chosen from baker's or brewer's yeast extracts.

20. An agent according to claim 5, in which the yeast extract is chosen from baker's or brewer's yeast extracts.
